(19) United States
(12) Patent Application Publication
(10) Pub. No.: US 2020/0129654 A1
(43) Pub. Date: Apr. 30, 2020

(54) Dressing Providing for the Controlled and Sustained Release of Metformin

(71) Applicant: Urgo Recherche Innovation et Developpement, Chenove (FR)

(72) Inventors: Claire Bouvier, Dijon (FR); Sébastien Very, Eperney sous Gevrey (FR)

(73) Assignee: Urgo Recherche Innovation et Developpement, Chenove (FR)

(21) Appl. No.: 16/629,132

(22) PCT Filed: Jul. 12, 2018
(86) PCT No.: PCT/FR2018/051758

§ 371 Date: Jan. 7, 2020

(30) Foreign Application Priority Data
Jul. 12, 2017 (FR) 17 56590

(51) Int. Cl. A61L 15/24; A61K 31/155; A61L 15/44

(52) U.S. Cl. CPC A61L 15/24; A61L 2300/412; A61L 15/44; A61K 31/155

(57) ABSTRACT

The present invention relates to a dressing providing for the controlled and sustained release of metformin.

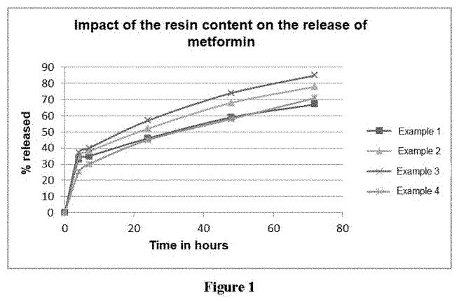

The present invention relates in particular to the use of a resin selected from the aromatic resins, to promote the release of metformin into an elastomeric matrix.

### SUMMARY

The present invention relates to the use of a resin as releasing agent for metformin.

The present invention relates in particular to the use of a resin selected from the aromatic resins to promote the release of metformin into an elastomeric matrix, as well as a composition containing same.

### PRIOR ART

The healing of wounds usually depends on the proliferation of new epithelial, endothelial and connective tissue. It therefore involves a set of intertwined events that are interconnected by successive and reciprocal inductions of the various cells involved. Each step is induced by the preceding step and can therefore only take place once the preceding step is complete. It involves a complex process underpinned by successive waves of growth factors and inflammatory mediators.

The pharmaceutical market currently offers many topical preparations recommended for promoting the healing of wounds. Indeed, their action results from the complementarity of the various products of which they are composed and which give them, within a certain limit, their healing properties. They protect the wounds from the surrounding environment through an antiseptic coating. They stimulate the development of vascularisation and regulate epidermalisation. These topical forms are mainly composed of a lipid mixture (lanolin, vaseline, glycerin . . . ) to which acids (salicylic, benzoic, malic), minerals (zinc oxide, titanium oxide) or halides (starch-iodide) are added. Some also contain collagen, fibrinogen, serum proteolytic enzyme (providing amino acids) or even vitamins (vitamin A) and hormones (4-chlorotestosterone acetate). There is also an ointment (Madecasol tulgras from SYNTEX Labs) the healing action of which is provided by the combination of a mixture of three triterpenes extracted from roots of the plant Centella asiatica (TCEA). These compounds stimulate the biosynthesis of collagen and glycoaminoglycans. Patent FR 2 809 310 describes the use of metformin in a topical composition having a healing and/or angiogenic effect. The different galenic formulations of envisaged compositions are in the form of an oil, cream, foam, liniment, lotion, ointment, liquid, gel, milk, powder or spray.

However, local application of active ingredient(s) in the form of topical compositions needs to be frequently repeated in order to ensure an effect on healing. However, for many wounds, it is necessary to apply a local treatment promoting healing over several days, or even several weeks, in order to obtain good healing and to avoid repetition of the treatment in order to limit the risk of infection linked to handling the compositions.

It would therefore be desirable to have a device, such as a dressing, comprising an active ingredient, in particular metformin, in order to effectively treat wounds over several days. For economic reasons, the dressing should ideally be applied for a duration of approximately 72 hours. Changing the dressing too frequently introduces additional costs with regard to the cost of care personnel required for changing said dressing. Changing too frequently can also increase the risk of infection. Indeed, at the time when the dressing is removed, the wound is exposed to the bacteria present in the environment.

However, when the active agent has a strong affinity for the exudates, as is the case for metformin, it has a tendency to be released too quickly onto the dressing, so that a peak concentration of the active agent is released by the dressing in the hours following its application and the beneficial effects of the treatment do not last for more than a few hours. On the contrary, the dressing should allow a controlled and continuous release of the active agent over several days, in particular for around three days. In particular, in order to attain a satisfactory response level at the receptors of the wound, a sufficient quantity of active agent must be released during the application of the device, then a weaker but continuous release of the active agent must be maintained throughout the duration of application of the device on the wound, in order to maintain the desired effects. This involves, in other words, administering a particular release profile involving a bolus in the time just after application of the formulation, then maintaining a lower level of release of active ingredient throughout the duration of the treatment.

### SUMMARY OF THE INVENTION

The present invention aims to address these problems by proposing a composition that is able to be used in a dressing type device comprising metformin, allowing a release thereof according to a continuous and controlled profile over several days.

Thus, the present invention has developed a specific composition made from ABA-type triblock polymers, comprising two thermoplastic styrene terminal blocks A and a central elastomeric block B which is a saturated olefin, allowing the preparation of an elastomeric matrix that is able to be used in a dressing, which can be in the form of a self-supporting dressing, said matrix allowing a release of metformin according to a continuous and controlled profile over several days.

More specifically, it has been discovered, and this constitutes the basis of the invention, that the use of an aromatic resin present in the composition in a predetermined amount by weight, allows improved release of metformin when compared with the dressing not containing said resin.

The use of specific resins makes it possible to increase the percentage of metformin released.

The addition of specific resins also makes it possible to reduce the viscosity of the elastomeric mixture and to mechanically strengthen the elastomeric matrix. This is particularly advantageous in the method for producing the elastomeric matrix. The reduced viscosity allows the composition to be coated more easily onto a framework. In the case of a self-supporting dressing, the improvement in cohesion of the elastomeric matrix makes possible a better moulding and demoulding thereof. In addition, due to the specific resins used, the production temperature of the composition can be reduced by approximately 10° C., which makes it possible to introduce components into the mixture which are sensitive to thermal treatments, such as active ingredients for example.

According to a preferred embodiment, the dressing according to the invention does not adhere to latex surgical gloves.

Thus, according to a first aspect, the present invention relates to a composition comprising:
5 to 20% by weight of at least one triblock copolymer of the ABA type, comprising two thermoplastic styrene terminal blocks A and a central elastomeric block B which is a saturated olefin or a mixture of triblock copolymers of the ABA type, comprising two thermoplastics styrene terminal blocks A and a central elastomeric block B which is a saturated olefin, based on the total weight of the composition
50 to 80% by weight of at least one plasticiser,
0.5 to 15% by weight of an alpha-methylstyrene resin, having a softening point between 80 and 125° C., preferably between 90 and 110° C.,
0.1 to 15% metformin,

the percentages being based on the total weight of the composition.

According to a second aspect, the present invention relates to an elastomeric matrix obtained from such a composition and an interface dressing, with substrate or self-supported, comprising said elastomeric matrix.

Finally, the invention also relates to a use of an alpha-methylstyrene resin for promoting the release of metformin in a composition, in particular used in a dressing.

### DETAILED DESCRIPTION

Elastomer

The composition according to the invention comprises at least one ABA triblock copolymer.

The block copolymers used in the context of the invention are ABA triblock copolymers comprising two thermoplastic styrene terminal blocks A and a central elastomeric block B which is a saturated olefin. The B blocks of saturated olefins are, for example, ethylene-butylene, ethylene-propylene or ethylene-ethylene-propylene blocks.

For the sake of simplicity, in the present description, the polymer blocks constituting the above-mentioned copolymers are designated by the nature of their recurrent units. Thus, the expression “block “or “styrene block A” designates a poly(styrene) block and the expression “block” or “saturated olefin block” designates a poly(saturated olefin) block.

According to an embodiment of the invention, the composition comprises a mixture of two copolymers, said mixture comprising at least one copolymer which has a viscosity between 0.01 and 1 Pa·s measured in a 5% mass/mass solution in toluene and at least one copolymer having a viscosity between 0.01 and 0.5 Pa·s measured in a 15% (mass/mass) solution in toluene.

Triblock copolymers with a saturated central block are well known to a person skilled in the art and are marketed, for example:
by KRATON under the name KRATON® G, and, in particular, the grades KRATON® G1651, KRATON® G1654, KRATON® G 1657, KRATON® G1652 or KRATON® G1650, and by KURARAY under the name SEPTON® and, in particular, the grades 8006 or 8004 for poly(styrene-ethylene-butylene-styrene) block copolymers (abbreviated as SEBS);
by KURARAY under the name SEPTON® for poly(styrene-ethylene-propylene-styrene) block copolymers (abbreviated as SEPS) and in particular the grades 2005, 2006 or 2063 and for poly(styrene-ethylene-ethylene-propylene-styrene) block copolymers (abbreviated as SEEPS) and in particular the grades 4033, 4044, 4055, 4077 or 4099.

Copolymers having a viscosity between 0.01 and 1 Pa·s, measured in a 5% (mass/mass) solution in toluene, include the copolymers marketed by KRATON under the grades KRATON® G 1651 and KRATON® G 1654 and copolymers marketed by KURARAY under the grades SEPTON® 2005, 2006, 8006, 4055, 4077, 4044 or 4099.

Copolymers having a viscosity between 0.01 and 0.5 Pa·s, measured in a 15% (mass/mass) solution in toluene, include the copolymers marketed by KRATON under the grades KRATON® G 1650, KRATON® G 1657 and KRATON® G 1652 and copolymers marketed by KURARAY under the grades SEPTON® 2063 or 4033.

These viscosities are measured at 30° C. using a Brookfield model LVI viscometer in a 5% or 15% (mass/mass) solution in toluene, depending on the molecular weight of the copolymer. These viscosities are measured at 30° C. using a Brookfield model LVI viscometer in a 5% or 15% (mass/mass) solution in toluene, depending on the molecular weight of the copolymer.

In the framework of the present invention, SEBS, SEPS or SEEPS triblock copolymers having a styrene content between 25 and 45% by weight based on the weight of said SEBS, SEPS or SEEPS copolymer are preferred.

According to a preferred embodiment, the composition comprises a single ABA triblock copolymer, and more preferably a single elastomeric block copolymer.

In general, the quantity of copolymers in the final composition can be between 5 and 20% by weight, preferably between 7 and 15% by weight, based on the total weight of the composition.

The Resins

The resins used in the composition according to the invention are aromatic hydrocarbon resins, in other words made uniquely from aromatic monomers. They differ from aliphatic resins, made uniquely from aliphatic monomers, or aliphatic/aromatic resins, made from aliphatic and aromatic monomers. Without wishing to be bound by any particular theory, it appears that these resins have a good solubility in the block A of the ABA copolymers and strengthen this styrene block, which improves the cohesion of the final elastomeric matrix obtained.

Indeed, it has been discovered that the use of an aromatic resin present in the composition in a predetermined amount by weight, allows improved release of metformin when compared with the dressing not containing said resin. This was particularly unexpected given that the resin contributes to the cohesion of the elastomeric matrix. It was therefore initially expected that the metformin would be trapped in the elastomeric matrix by the addition of such a resin. However, on the contrary, the resin facilitates and improves the release of metformin. This may have an economic advantage, since it is possible to use a smaller quantity of metformin, which is a relatively expensive compound, in the composition, for a same released quantity. The extended profile of metformin release is also beneficial.

The aromatic monomer is, in particular, alpha-methylstyrene. Thus, according to a particularly preferred embodiment, the aromatic hydrocarbon resin is chosen from the homopolymer and copolymer resins of alpha-methyl-styrene.

Among the aromatic resins tested, a certain number were not entirely satisfactory. Indeed, some grades of resin, due to their high softening point, needed to be heated to high temperatures (greater than 140° C.) in order to produce the composition of the invention. When working at such temperatures, there is a risk of the plasticiser evaporating. When hydrocolloids (such as carboxymethyl cellulose) or active ingredients are added to the composition, these may degrade.

Thus, the resins used in the compositions according to the invention are alpha-methylstyrene resins having a softening point in the range between 80 and 125° C., preferably between 90 and 110° C.

The softening point is measured according to standard ISO 4625 (“Ring and Ball” method).

Preferably, the resin according to the invention is an alpha-methylstyrene resin having a softening point in the range between 95 and 105° C. or between 115 and 125° C. or a poly(styrene-co-alpha-methylstyrene) resin having a softening point between 95° C. and 115° C.

The preferred resins above are well known to a person skilled in the art and are commercially available, sold for example under the following tradenames:
Sylvares SA 100 and Sylvares SA 120 from Arizona Chemical: alpha-methylstyrene resins having a softening point in the range between 95 and 105° C. or between 115 and 125° C. respectively,
Cleartack W90 or Norsolene W90 resin from Cray Valley: poly(styrene-co-alpha-methylstyrene) resin having a softening point between 85 and 95° C.,
the resins Kristalex 3100LV, Kristalex F100, Kristalex 3105SD and Kristalex F115 from Eastman: poly(styrene-co-alpha-methylstyrene) resins having a softening point of 100° C., or between 96 and 104° C. or of 105° C., or between 114 and 120° C. respectively.

According to a preferred embodiment, the compositions according to the invention comprise no resin other than the previously described alpha-methylstyrene and, in particular, do not comprise tackifying or sticky resin.

In the context of the present invention, the resin is preferably present in a quantity of 0.5 to 15%, more preferably 2 to 10% by weight, based on the total weight of the composition.

The Plasticiser

In order to produce interface dressings, the mixture of copolymers and the resin in the composition according to the invention are associated with one (or more) plasticiser compounds.

Suitable plasticisers for use here are well known and intended to improve the stretching, flexibility, extrusion or usage properties of copolymers. If necessary, one or more plasticisers can be used for this purpose.

In general, preferred plasticisers are liquid compounds, compatible with the central saturated olefin block of the above-mentioned block copolymers.

Plasticisers suitable to be used for this purpose include, in particular, plastifying mineral oils.

Alternatively, it is also possible to use synthetic products made from saturated hydrocarbon liquid mixtures such as, for example, the products marketed by TOTAL under the name GEMSEAL® and in particular the product GEMSEAL® 60 which is an isoparaffinic mixture from a fully hydrogenated petroleum fraction.

In the context of the present invention, plasticising oils are preferably used, and in particular mineral oils formed of paraffin or naphthenic compounds, or mixtures thereof, in variable proportions.

Particularly preferred plasticising mineral oils are formed of mixtures of paraffin and naphthenic compounds, and in particular mixtures in which the proportion of paraffin compounds is in the majority.

Particularly suitable plasticising oils include the products marketed by SHELL under the names ONDINA® and in particular ONDINA® 919 or the oil marketed by PETRO CANADA under reference PURETOL® 9D or the oil BLANDOL marketed by Sonneborn, or again the oil Pionier 2076P marketed by Hansen & Rosenthal.

In addition to oils, the plasticiser may also comprise vaseline. The vaseline used in the compositions of the invention is a commercially available vaseline complying with the French Pharmacopoeia.

In the context of the present invention, the vaseline is present in a quantity of 1 to 30%, preferably 5 to 25% by weight, based on the total weight of the composition. In the context of the present invention, the plasticiser is present in a quantity of 50 to 80%, preferably 60 to 70% by weight, based on the total weight of the composition.

The plasticiser is preferably composed of a mixture of mineral oil and vaseline, the mineral oil being present in a quantity ranging from 45 to 60% by weight based on the total weight of the composition, the vaseline being present in a quantity ranging from 5 to 20% by weight based on the total weight of the composition.

Metformin

Metformin is an oral anti-diabetic of the family of normoglycemic biguanides used in the treatment of type II diabetes. Its role is to reduce the insulin resistance of the organism that is intolerant to carbohydrates and to reduce hepatic neoglucogenesis. Metformin is administered orally. Metformin is absorbed in the small intestine, circulates in the blood in a non-attached manner and is excreted, unchanged, via the kidneys. Its mechanism of action is complex and is not currently fully understood. Metformin is a normoglycemic agent: it does not act on insulin secretion or on the sensitivity of glucose-using tissues to insulin (muscles, adipose tissue). Metformin also has a role in the inhibition of neoglucogenesis, by inhibiting mitochondrial glycerophosphate deshydrogenase, and in the membrane transport of glucose (reducing intestinal reabsorption). It also increases the release of glucagon-like peptide-1, inhibits the glucagon route and increases the production of lactates by the enterocytes.

According to a preferred embodiment, the metformin used is in the form of a metformin hydrochloride.

The particle size of the metformin, when calculated according to the Fraunhoffer optical model between 0.375 μm and 2000 μm, measured by laser with dry powder module, preferably has the following characteristics:
d50<300 μm, preferably d50<200 μm
d90<600 μm, preferably d90<510 μm.

In the context of the present invention, the particle size distribution of the metformin is preferably unimodal.

In the context of the present invention, the quantities of metformin introduced into the dressings are 0.5 to 15% by weight, preferably 1 to 10% by weight, based on the total weight of the elastomeric matrix.

Additional Active Ingredient:

In addition to metformin, the composition according to the invention may comprise at least one other active ingredient for inducing or accelerating healing, or having a favourable role in the treatment of the wound.

These active substances include, in particular, by way of example:
agents promoting healing, such as retinol, vitamin A, vitamin E, N-acetyl hydroxyproline, extracts of Centella asiatica, papain, silicone, essential oils of thyme, niaouli, rosemary and sage, hyaluronic acid, potassium sucrose octasulfate, sucralfate and allantoin;
antibacterial agents, such as silver salts or complexes (such as silver sulfates, silver nitrates, silver sulfamides or even silver-based zeolites), zinc or copper salts, metronidazole, neomycin, penicillins, clavulanic acid, tetracyclines, minocycline, chlortetracycline, aminoglycosides, amikacin, gentamicin and probiotics;
antiseptics, such as chlorhexidine, trichlosan, biguanide, hexamidine, thymol, lugol's iodine, povidone-iodine, benzalkonium and benzethonium chloride;
painkillers, such as paracetamol, codeine, dextropropoxyphene, tramadol, morphine and its derivatives, corticosteroids and their derivatives;
local anaesthetics, such as lidocaine, benzocaine, dibucaine, pramoxine hydrochloride, bupivacaine, mepivacaine, prilocaine and etidocaine;
and anti-inflammatories, such as nonsteroidal anti-inflammatory drugs (NSAIDs), aspirin or acetylsalicylic acid, ibuprofen, ketoprofen, flurbiprofen, diclofenac, aceclophenac, ketorolac, meloxicam, piroxicam, tenoxicam, naproxen, indomethacin, naproxcinod, nimesulide, celecoxib, etoricoxib, parecoxib, rofecoxib, valdecoxib, phenylbutazone, niflumic acid and mefenamic acid.

Of course, the composition according to the invention may also comprise one or more other compounds known for their action in the cleaning phase such as, for example:
enzymes;
urea.

The agent promoting healing is preferably selected from the healing promoting agents retinol, vitamin A, vitamin E, N-acetyl hydroxyproline, extracts of Centella asiatica, papain, silicone, essential oils of thyme, niaouli, rosemary and sage, hyaluronic acid, potassium sucrose octasulfate, sucralfate and allantoin.

The composition according to the invention comprises 0.1 to 15% of additional active ingredient, preferably 1 to 10% by weight, based on the total weight of the composition.

Hydrocolloid

According to a particularly preferred embodiment of the invention in the context of producing interface dressings that are self-supporting, have a substrate or have a framework for the healing of wounds, the compositions according to the invention comprise hydrophilic particles of a hydrocolloid (or hydrocolloid particles).

These particles enable the painless withdrawal of an interface dressing and its retention on the wound in a humid environment, in order to promote healing.

To this effect, a small quantity of hydrophilic particles of a hydrocolloid is thus either deposited on the surface of the elastomeric matrix once this is formed or, preferably, dispersed homogeneously within the composition according to the invention.

Here, “hydrocolloid” or “hydrocolloid particles” shall mean any compound regularly used by a person skilled in the art for its ability to absorb aqueous liquids such as water, physiological serum or the exudates of a wound.

Suitable hydrocolloids include, for example, pectin, alginates, natural plant gums such as, in particular, gum karaya, cellulose derivatives such as carboxymethyl celluloses and their salts of alkali metal salts such as sodium or calcium, as well as synthetic polymers based on acrylic acid salts, known under the name “superabsorbents” such as, for example, the products marketed by CIBA Specialty Chemicals under the name SALCARE® SC91 as well as mixtures of these compounds.

Certain of these superabsorbents, termed “microcolloids” because they have a particle size less than 10 micrometres, can of course also be used.

The preferred hydrocolloids in the context of the present invention are alkali metal salts of carboxymethyl cellulose and, in particular, sodium carboxymethyl cellulose (CMC).

The hydrocolloid particle size is generally between 50 and 100 microns, advantageously of order 80 microns.

In general, the quantity of hydrocolloid particles incorporated in the composition according to the invention is advantageously less than or equal to 25% by weight, advantageously of order 2 to 20% by weight, preferably 5 to 18% by weight, more preferably 10 to 15% by weight, based on the total weight of the composition.

If the hydrocolloid particles are deposited at the surface of the elastomeric matrix once this is formed, their quantity will preferably be of order 1 to 10% and more particularly from 2 to 5% by weight, based on the total weight of the elastomeric matrix.

The selection of a quantity of hydrocolloid particles within these ranges of values is important for the production of an interface dressing and, in particular, a self-supporting aerated interface dressing, in order to avoid gelling of the composition resulting in closure of the through-holes during the absorption of exudates.

Tackifying Resins

The composition according to the invention can also comprise at least one tackifying resin in order to give it an adhesive property facilitating its positioning on the wound.

The tackifying resins that can optionally be used in the compositions according to the invention are chosen, in particular, from low molecular weight polyisobutylenes. In general, the use of hydrogenated resins such as Escorez® resins from the series 5000 are preferred, and yet more preferably the resin Escorez 5380®.

Antioxidants:

The composition according to the invention may also comprise antioxidants.

Here “antioxidants” shall mean compounds commonly used by a person skilled in the art to ensure the stability of compounds in the formulation of compositions, in particular with respect to oxygen, heat, ozone or ultraviolet radiation.

Examples of suitable antioxidants include, in particular, phenolic antioxidants such as, in particular, the products marketed by BASF under the names IRGANOX® 1010, IRGANOX® 565 and IRGANOX® 1076.

In general, these antioxidants can be used alone or in combination in a quantity of order 0.05 to 1% by weight, preferably 0.05 to 0.2% by weight, based on the total weight of the composition.

In the context of the present invention, the use of the product IRGANOX® 1010 is preferred in a quantity between 0.05 and 0.2% by weight, based on the total weight of the composition.

Additive:

Additives that are suitable for use in the compositions according to the invention include known compounds for promoting the release of active agents, such as, for example, the products Montanox® 80 or Sepinov® EMT 10 (copolymer of the 2-methyl-2[(1-oxo-2-propenyl)amino]-1-propanesulfonic acid salt and propenoic acid 2-hydroxyethyl ester or the mixture of 2-octyl-1-dodecanol, D-xylopyranoside, 2-octyldodecyl and polyethyleneglycol 30 dipolyhydroxystearate) which are commonly used in URGOTUL® products which incorporate active agents.

These additives can be used in a quantity of order 0.01 to 10% by weight, preferably 0.05 to 5% by weight, based on the total weight of the elastomeric matrix.

Clearly, the particular embodiments which have just been described can be implemented separately or according to any of their combinations.

The compositions according to the invention make it possible, in particular, to produce self-supporting interface dressings or interface dressings having a framework or a substrate.

In the context of producing an interface dressing, it is preferable to use a composition which comprises compounds (copolymers, mineral oil, vaseline, antioxidant and hydrocolloids) of the same type as, or identical to, those used in the product URGOTUL®.

Elastomeric Matrix

In order to produce a dressing, the compositions according to the invention are formed as a thin layer, with through-holes, preferably arranged in a distributed manner in said layer in order to form an elastomeric matrix.

The invention also concerns, according to another aspect, an elastomeric matrix obtained from a composition according to the invention such as described above.

The through-holes can be produced by perforating or punching a composition according to the invention, formed beforehand as a thin layer, alone or combined with a temporary substrate or a protective film commonly used for dressing manufacture, or even by a woven coating on a temporary substrate.

Alternatively, the polymer matrices according to the invention can be produced by hot casting of a composition as described above, on a plate etched with the pattern selected for forming the through-holes, followed by cooling and demoulding.

In general, the polymer matrices according to the invention have a thickness between 0.4 mm and 2 mm, preferably between 0.5 mm and 1 mm, more preferably of order 0.6 to 0.7 mm.

The through-holes can be of any geometry and will have, for example, a circular, rectangular, trapezoidal or square cross-section.

The surface area is generally between 1 and 7 mm².

These holes are preferably distributed in a regular manner, with a density such that the total surface area of the holes is between 20 and 70%, preferably between 30 and 60%, of the total surface area of the dressing.

According to a preferred embodiment, the polymer matrix, when it is used in a preferably self-supporting interface dressing, has the form of an aerated net (or grid), preferably with square mesh, having:
a thickness of the net between 0.4 and 2 mm;
a “thread width” (width of the space between two consecutive holes) between 1 and 10 mm, and preferably between 1 and 5 mm;
a grammage between 200 and 1700 g/m², and preferably between 300 and 800 g/m².

According to a particularly preferred embodiment of the invention, such an elastomeric matrix is in the form of an aerated net with square mesh, having:
a thickness of the net of approximately 750 microns;
a thread thickness (or mesh size) of order 0.8 mm;
a grammage of order 390 g/m².

In order to produce such elastomeric matrices, further details can be found in patent application FR 2 936 158.

It may also be envisaged to use this elastomeric matrix for coating a framework or a substrate.

The techniques for producing an interface dressing with frame or with substrate are also well known to a person skilled in the art and reference can be made, for example, to the methods described in patent applications WO 00 16725 and FR 2 936 159 or WO 2015/018720.

According to a preferred embodiment, the elastomeric matrices obtained by means of compositions according to the invention are not sticky, in other words they have an adhesive power to the skin, determined according to method EN 1939, of less than 40 cN/cm, and preferably less than 35 cN/cm. A substrate sample of width 20 mm and length 150 mm is placed on the forearm. After 10 minutes, the adhesive power is measured using a dynamometer at a traction speed of 900 mm/min with an angle of 90°.

Dressing

The invention also relates, according to a preferred embodiment, to an interface dressing characterised in that it comprises an elastomeric matrix as described above.

According to an alternative embodiment, the present application is intended to cover a self-supporting interface dressing comprising an elastomeric matrix in the form of a thin layer having through-holes for allowing the passage of exudates, obtained from a composition comprising:
5 to 20% of at least one styrene-saturated olefin-styrene triblock copolymer,
50 to 80% by weight of at least one plasticiser,
0.5 to 15% of at least one alpha-methylstyrene resin, having a softening point in the range between 80 and 125° C., preferably between 90 and 110° C.,
0.1 to 15% of at least metformin,

the percentages being based on the total weight of the composition.

Preferably, the interface dressing according to the invention does not adhere to latex gloves. To achieve this, the composition may preferably comprise:
for 100 parts by weight of a mixture P of two specific triblock copolymers of the type styrene-saturated olefin-styrene, a first which has a viscosity between 0.01 and 1 Pa·s as measured in a 5% (mass/mass) solution in toluene and a second which has a viscosity between 0.01 and 0.5 Pa·s as measured in a 15% (mass/mass) solution in toluene;
300 to 1000 parts by weight of a plasticiser H, preferably an oil plasticiser; and
90 to 600 parts by weight of vaseline V;

it being also specified that:
the total quantity, represented by P+H+V, of mixture of elastomers, plasticiser and vaseline is between 490 and 1700 parts by weight;
the ratio between the total quantity of the mixture of elastomer, plasticiser and vaseline and the quantity of vaseline, represented by P+H+V/V, is less than 11;

said mixture of two copolymers comprises at least 20% by weight of the first copolymer,

the composition also comprising 0.5 to 15% by weight of an alpha-methylstyrene resin, having a softening point between 80 and 125° C., preferably between 90 and 110° C., and 0.1 to 15% by weight of metformin,

the percentages being based on the total weight of the composition.

In order to protect the composition from the external environment, the interface dressing can be covered, preferably on each of its faces, by a temporary protective film which will be removed before use by the user.

In order to further facilitate the handling of the interface dressing, in particular if it is self-supporting, the two temporary protective films can be substituted by a single protection such as described in patent application WO2008/145884 or in patent application WO2015/018720, the structure of which particularly facilitates the application of the dressing on the wound.

### FIGURE

The FIGURE is a graph showing the dissolution profiles, evaluated in percentage of metformin released based on the quantity contained in the mass based on the dressings described in example 1 and example 4.

The present invention is illustrated in the non-limiting examples presented below.

### EXAMPLES

Preparation of the Compositions

The compositions of examples 1 to 4 have been developed using the following constituents in the proportions, expressed in percentage by weight, listed in table 1 below.

Elastomer: block copolymer of poly(styrene-ethylene-butylene-styrene) (abbreviated as SEBS):
KRATON® G 1651 or Kraton G 1654 ES

Plasticiser: mineral oil Pionier 2076P marketed by Hansen & Rosenthal

Vaseline: vaseline Codex® A marketed by AIGLON

Antioxidant: IRGANOX® 1010 marketed by BASF

Hydrocolloid: Sodium carboxymethyl cellulose CMC BLANOSE® 7H4XF marketed by ASHLAND,

Resins: Sylvares SA 100, alpha-methylstyrene resin having a softening point in the range between 95 and 105° C., marketed by Arizona Chemical

Active ingredient: Metformin from IPCA

Production of the Composition

The carboxymethyl cellulose, metformin and SEPINOV EMT 10 are premixed and sieved to 400 μm.

The vaseline and oil are introduced into a mixer at a setpoint temperature of 90° C. and a speed of 60 to 70 rotations per minute (rpm), then the sieved carboxymethyl cellulose, SEPINOV EMT 10 and metformin powders are introduced. Mixing takes place for 5 minutes.

The setpoint temperature is increased to 140° C. The elastomer and antioxidant are then introduced. Mixing takes place for 45 minutes (until a smooth and homogeneous mixture is obtained). The resin is introduced 15 minutes before the end.

Following this, the mixer is left to cool and then drained.

The compositions thus obtained were coated on a weave (marquisette 601 marketed by MDB Texinov) according to the methods described in patent applications WO 00 16725 and FR 2 936 159 or WO 2015/018720

Method for Measuring the Metformin Release:

Procedure for the dissolution test:

Kinetic release of metformin and potassium sucrose octasulfate by the dissolution test method under the following conditions:
USP instrument 6, equipped with rotating cylinders
agitation speed: 60 RPM
temperature of the medium: 32.0° C.±0.5° C.
medium:

physiological serum
volume of the medium: 1000 mL
sampling of the medium at time 0 h, 4 h, 7 h, 24 h, 48 h and 72 h,
surface area of the dressings: 100 cm²

The dressings are weighed before being placed on the cylinders.

The results are expressed in % of metformin or potassium sucrose octasulfate released, taking account of the theoretical quantity of metformin or potassium sucrose octasulfate in each dressing, i.e.:

Current mass of the dressing x content of metformin or potassium sucrose octasulfate.

The quantity of metformin or potassium sucrose octasulfate of each sample is determined by:

HPLC/UV

Apparatus

Column: Luna SCX 5 μm−100 Å−100 mm×4.6 mm, ref. Phenomenex 00D-4398-E0,

HPLC system equipped with a UV detector, a sample changer and a column oven.

Reagents

Ammonium dihydrogen phosphate for analysis. For example: ref. VWR 21305.290 or equivalent,

Low UV acetonitrile for analysis. For example: ref. VWR 20048.290 or equivalent, Sodium chloride for analysis. For example: ref. VWR 27810.295 or equivalent,

Ultra pure water.

Reference Substance

Secondary reference metformin hydrochloride preserved in a closed flask, stored in a desiccation cabinet.

Potassium sucrose octasulfate preserved in a closed flask, stored in a desiccation cabinet.

Solutions/Samples to be Prepared

Mobile Phase

The mobile phase is a solution of ammonium dihydrogen phosphate at 17 g/L to which is added 2% of acetonitrile.

Test Solutions

Preliminary precaution: the physiological serum used during the analysis will be thermostatically heated beforehand to 32° C.

Procedure:

TABLE 1
Chromatographic conditions

| | |
|---|---|
| Column | Luna SCX - 5 μm - 100 mm × 4.6 mm |
| Mobile phase | Ammonium dihydrogen phosphate at |
| | 17 g/L + 2% acetonitrile |
| Flow rate | 1.2 mL/min |
| Temperature of sample | Not refrigerated and not temperature |
| changer | monitored |
| Temperature of the | 35° C. |
| column oven | |
| Mode | Isocratic |
| Injection volume | 10 μL |
| Detection | UV adjusted to 232 nm |
| Retention time of the | Approximately 6 minutes |
| metformin peak | |
| Analysis time | 10 minutes |

Reporting of the Results

All results are reported in summary tables such as those described above, then the release kinetics of the metformin are monitored as a function of time (expressed in hours).

### Examples 1 to 4

| | Ex. 1 | Ex. 2 | Ex. 3 | Ex. 4 | Ex. 5 | Ex. 6 |
|---|---|---|---|---|---|---|
| Tradename | % weight | | | | | |
| SEBS (Kraton G1651 ES from | 4.9 | 4.9 | 4.9 | 4.9 | | |
| Kraton Polymer) | | | | | | |
| SEBS (Kraton G1654 ES from | | | | | 6.0 | 6.0 |
| Kraton Polymer) | | | | | | |
| White mineral oil (Pionier 2076P) | 69.8 | 67.8 | 60.2 | 49.8 | 62.4 | 52.8 |
| Sodium carboxymethyl cellulose | 15.0 | 15.0 | 15.0 | 15.0 | 14.0 | 14.0 |
| (CMC Blanose 7H4XF from | | | | | | |
| Aschland) | | | | | | |
| Pentaerythritol tetrakis3-(3,5-di- | 0.2 | 0.2 | 0.2 | 0.2 | 0.12 | 0.12 |
| tert-butyl-4- | | | | | | |
| hydroxyphenyl)propionate | | | | | | |
| (Irganox 1010 from BASF) | | | | | | |
| Vaseline (Vaseline Codex A from | 5.0 | 5.0 | 5.0 | 5.0 | 5.0 | 5.0 |
| Synteal) | | | | | | |
| Alpha-methylstyrene resin having | 0 | 2.0 | 9.6 | 20 | 0 | 9.6 |
| a softening point in the range | | | | | | |
| between 95 and 105° C., Sylvares | | | | | | |
| SA 100 | | | | | | |
| Copolymer of 2-methyl-2[(1-oxo- | 0.1 | 0.1 | 0.1 | 0.1 | 5.0 | 5.0 |
| 2-propenyl)amino]-1- | | | | | | |
| propanesulfonic acid salt and | | | | | | |
| propenoic acid 2-hydroxyethyl | | | | | | |
| ester (Sepinov EMT 10 from | | | | | | |
| SEPPIC) | | | | | | |
| Metformin from IPCA | 5.0 | 5.0 | 5.0 | 5.0 | | |
| Potassium sucrose octasulfate | | | | | 7.5 | 7.5 |
| from Euticals | | | | | | |
| Marquisette | 601 | 601 | 601 | 601 | 601 | 601 |

TABLE 3
metformin release in percentage released based
on the quantity contained in the mass

| | Average cumulative release in percent | | | | |
|---|---|---|---|---|---|
| Examples | 4 h | 7 h | 24 h | 48 h | 72 h |
| Example 1 | 33 | 35 | 46 | 59 | 67 |
| Example 2 | 35 | 38 | 52 | 68 | 78 |
| Example 3 | 37 | 40 | 57 | 74 | 85 |
| Example 4 | 25 | 30 | 45 | 58 | 71 |
| Example 5 | 13 | 15 | 16 | 16 | 16 |
| Example 6 | 11 | 13 | 15 | 15 | 15 |

It is observed that at 2% and 9.6% by weight, the alpha-methylstyrene resin increases the percentage of released metformin. At 20% of said resin, approximately the same proportions of released metformin are found as when the composition comprises no resin.

For the maquettes of examples 5 and 6 containing potassium sucrose octasulfate, no difference in release is observed when the alpha-methylstyrene is added.

1. A composition comprising
5 to 20% by weight of at least one triblock copolymer of the ABA type, comprising two thermoplastic styrene terminal blocks A and a central elastomeric block B which is a saturated olefin or a mixture of triblock copolymers of the ABA type, comprising two thermoplastics styrene terminal blocks A and a central elastomeric block B which is a saturated olefin, based on the total weight of the composition
50 to 80% by weight of at least one plasticiser,
0.5 to 15% by weight of an alpha-methylstyrene resin, having a softening point between 80 and 125° C., preferably between 90 and 110° C.,
0.1 to 15% metformin,
the percentages being based on the total weight of the composition.

2. The composition according to claim 1, wherein the plasticiser is composed of a mixture of mineral oil and vaseline.

3. The composition according to claim 1, wherein it comprises hydrocolloid particles in a quantity less than or equal to 25% by weight, based on the total weight of the composition.

4. An elastomeric matrix, obtained from a composition according to claim 1, by formation of a thin layer and compression, or by hot casting of said composition on a substrate or a weave.

5. The elastomeric matrix according to claim 4, wherein it comprises a framework or a substrate, or in that it is self-supporting.

6. The self-supporting elastomeric matrix according to claim 5, wherein it comprises through-holes.

7. The self-supporting elastomeric matrix according to claim 5, wherein it is in the shape of an aerated net with a mesh size of order 4 mm² thickness of order 800 microns and grammage of order 400 g/m².

8. An interface dressing, wherein it comprises an elastomeric matrix according to claim 5.

9. A method for promoting the release of metformin in a composition, optionally implemented in a dressing comprising contacting the composition with an alpha-methylstyrene resin.

10. The method according to claim 9, wherein the alpha-methylstyrene resin has a softening point between 80 and 125° C.
